(19) United States
(12) Patent Application Publication
(10) Pub. No.: US 2011/0060357 A1
(43) Pub. Date: Mar. 10, 2011

(54) DEVICE FOR CUTTING OUT AND REMOVING CYLINDERS OF TISSUE FROM A TISSUE AND THE USE THEREOF

(75) Inventor: Joachim Reppenthien, Sauerlach (DE)

(73) Assignee: WISAP Gesellschaft fuer wissenschaftlichen Apparat ebau mbH, Sauerlach (DE)

(21) Appl. No.: 12/528,383

(22) PCT Filed: Feb. 22, 2008
(86) PCT No.: PCT/EP08/01406

§ 371 Date: Sep. 15, 2009

(30) Foreign Application Priority Data
Feb. 22, 2007 (DE) 10 2007 008 751.0
Oct. 17, 2007 (DE) 10 2007 049 796.4

(51) Int. Cl. A61B 17/3205

(52) U.S. Cl. 606/170

(57) ABSTRACT

The invention relates to a device for cutting out and removing cylinders of tissue from a tissue, which is located inside a body or joint cavity and/or in or on a wall region thereof, comprising a cutting device (12) with a hollow-cylindrical base body (14), a distal opening (18) on a distal end (16) of the base body (14), and a cutting element (20) surrounding the distal opening (18), further an actuating device (22) for rotating the base body (14) about the longitudinal axis (24) thereof on a proximal end (26), and/or on a region (28) facing the proximal end (26), of the base body (14), and a sealing device (30) for closing and opening a proximal opening (32) on the proximal end (26) of the body (14), and the use thereof.

The invention relates to a device for excising and removing cylinders of tissue from tissue located within a body cavity or articular cavity and/or in a wall area of same, and also to its use.

Such devices are generally known. These devices have, however, been shown to be decidedly disadvantageous in practice. Such devices are used, together with a trepan or a trepan sleeve, for example in the medical field of gynaecology for the removal of tissue from a uterus, the removal of a complete uterus or the treatment of myoma, etc. For example, such a device is known from EP 0 522 125 B1. Such devices are furthermore used in medicine in many, sometimes, different, other technical fields, such as for surgery in the abdominal cavity, on the stomach and for the excision of gallbladders or appendices. The disadvantage with all these devices is their overall design. In the main, such devices have a relatively expensive construction which is neither very compact nor stable. A particular disadvantage with all devices of this type is that these devices, after excising or punching out a cylinder of tissue, have to be withdrawn, together with a further separate instrument, whose use is at present indispensable, especially a gripper or other cutting instrument such as a claw gripper or myoma drill, etc., through the trepan sleeve in order to remove the cylinder of tissue from the area of the operation. As a consequence, the application of such devices is generally decidedly complicated, not very gentle for the patient and is time consuming. Finally, the continuous insertion and subsequent withdrawal of such devices into and out of the trepan sleeve poses an increased risk for the patient, for example due to traumatizing of the surrounding tissue during the removal of the cylinders of tissue and related effects.

The object of the invention is therefore to provide a device for excising and removing cylinders of tissue from tissue, with which the above disadvantages can be prevented, which is furthermore of particularly simple design and is compact and stable, and also can be cheaply manufactured, which is particularly reliable and at the same time enables a simple and fast removal of an excised or stamped out cylinder of tissue from the area of the operation, ensures a markedly easy and gentle, and particularly time-saving, use and also guarantees a substantially improved and increased patient safety while still enabling this device to be advantageously applied.

With regard to the device, this object is technically achieved in a surprisingly simple manner by the features of Claim 1.

Accordingly, by means of the design of the device according to the invention for excising and removing cylinders of tissue from tissue located within a body cavity or articular cavity and/or in a wall area of same, which includes a cutting device with a hollow cylindrical main body, a distal opening at a distal end of the main body and a cutting element surrounding the distal opening, an actuating device at a proximal end and/or at an area of the main body facing towards the proximal end for rotating the main body about the longitudinal axis of said main body and a sealing device for closing and opening a proximal opening at the proximal end of the main body, a simple, compact and stable construction of the overall device can be achieved, which is at the same time particularly cost-effective and reliable. Of particularly great importance in this case is that the structural web(s) or recess(es) provided serve as additional sealing lips or sealing barriers. At the same time this/these web(s) or recess(es) enable(s) centring relative to each other or relative to the housing element of the housing. In a particularly advantageous manner, this/these web(s) or recess(es) provide(s) additional security for both sealing elements with respect to each other and with respect to the housing element of the housing. Slipping or other displacement of both sealing elements from the installed position to an unwanted deformed position in which the sealing effect can no longer be maintained is thus excluded. Furthermore, the device according to the invention, which can also be called a morcellator, has the essential advantage that the device does not have to be withdrawn through the trepan sleeve together with another instrument, especially a gripping or other cutting instrument, such as a claw gripper or myoma drill, etc., for removal of the cylinder of tissue from the operating area after the cylinder of tissue has been excised or stamped out. Furthermore, the device according to the invention enables the user to extract morcellated tissue and at the same time leave the device itself in the operating area. This results not least in a noticeably easy, very gentle and especially time-saving operation of the device in general. Finally, the device according to the invention enables a substantially improved patient safety to be achieved.

Further advantageous details of the device according to the invention are described in Claims 2 to 40.

Furthermore, it is within the framework of the invention that the sealing device according to Claim 2 has a housing and at least one sealing element that can be fitted in the housing.

In an advantageous manner, the housing according to the features of Claim 3 has a housing element of essentially pot-shaped design that accommodates at least one sealing element and is mounted on the main body and a cover- or cap-shaped closure element that can be attached to the housing element.

The design measures of Claim 4 provide, in a quite advantageous manner, a simple, fast, reliable, and particularly cost-effective manufacture and assembly. Accordingly, the housing element and the closure element of the housing are releasably or unreleasably connected to each other by a threaded connection or a latching or snap-in connection. The constructive design of providing a releasable or unreleasable connection between the housing element and closure element of the housing in this case depends essentially on the type of use and purpose of the device according to the invention, i.e., whether the device is designed to be used once or several times. This in turn, and also the other way round, substantially affects the overall design of the device and the choice of materials of which the individual components of the device according to the invention are made.

If, for example, the device is meant just for single use, i.e., completely disposed of after use, the housing element and the closure element of the housing according to Claim 5 can be unreleasably connected to each other preferably by a threaded part with an adhesive agent, particularly a silicone adhesive, arranged in-between. For multiple use, the housing element and the closure element of the housing, which are then obviously made of metal, are on the other hand merely screwed together by means of a threaded part.

As an alternative to the threaded connection, a latching or snap-in connection can be provided, by means of which the housing element and the closure element of the housing according to Claim 6 are connected to each other through at least one latching projection or similar latching lug on the housing element and at least one further latching projection or similar latching lug on the closure element, which interact with each other and can be brought into mutual engagement. For a kinematic reversal, it is equally possible to provide the at least one latching projection or similar latching lug on the closure element and the at least one latching recess or similar on the housing element. Instead of the at least one further latching projection, at least one latching recess or similar can equally be provided, which interacts with the at least one latching projection.

For single use, the device according to the invention is in this connection advantageously designed according to the features of Claim 7. Accordingly, the at least one latching projection or similar latching lug on the housing element or on the closure element and the at least one further latching projection or similar latching lug on the closure element or on the housing element are designed in such a way that the housing element and the closure element of the housing are either on one hand unreleasably connected to each other after engagement of the at least one latching projection or similar latching lug and the at least one further latching projection or similar latching lug or on the other hand cannot be connected to each other after the at least one latching projection or similar latching lug and the at least one further latching projection or similar latching lug have been disengaged from each other. With the latter arrangement, it would, for example, be conceivable to provide one of the two latching projections or similar latching lugs with a design breakpoint, so that this latching projection or similar latching lug breaks away from the housing element or from the closure element if a certain tensile force is applied. Instead of the at least one further latching projection, at least one latching recess or similar, which interacts with the at least one latching projection, can equally be provided.

Of decidedly great importance are, furthermore, the design measures of Claim 8, according to which the sealing device includes two sealing elements which are arranged adjacent to each other in the housing on the longitudinal axis of the main body of the cutting device and interact with each other.

Moreover, it is provided according to the invention that one of the two sealing elements of the sealing device according to Claim 9 is essentially designed in the shape of a cone, a truncated cone, a pot, a hat or similar, has a slotted opening that is arranged in a plane which is offset in the direction of the distal end of the main body of the cutting device with respect to a plane extending through the base of the sealing element, and a circumferential section extending radially outwards in the plane that extends through the base of the sealing element, for fixing in the housing. Therefore the one of the two sealing elements is forced into its closed position and held there, due to its shape and the arrangement in the housing, in the opposing direction to the effect of a pressure artificially established within a body cavity or articular cavity, for example due to CO₂ insufflation. The sealing element is thereby pressed from the outside against another instrument or its shaft, for example a gripping or other cutting instrument, especially a claw gripper or myoma drill, to form a complete seal. Accordingly, it is ensured that the pressure, which is necessary for operating, i.e., for excising or punching out a cylinder of tissue from tissue and then removing the excised or punched out cylinder of tissue from the operating area, is maintained in any event. Consequently, an escape of gas, such as CO₂ or similar, from the body cavity or articular cavity is precluded and impedance of the removal of the cylinder of tissue or a traumatisation of the surrounding tissue during the removal of the cylinder of tissue is counteracted.

In particular, it is within the framework of the invention that one of the two sealing elements according to Claim 10 has at least two sealing lips, which are in mutual effective engagement to close and open the slotted opening.

Furthermore, another of the two sealing elements of the sealing device according to Claim 11 is designed in the form of a disc and has a central, approximately circular opening through which a further instrument, especially a gripping instrument or other cutting instrument, preferably a claw gripper or a myoma drill, can be inserted.

The central, approximately, circular, opening of the other of the two sealing elements preferably has, according to Claim 12, an internal diameter that is smaller or equal to an external diameter of the other instrument.

Furthermore, it is within the framework of the invention that the other of the two sealing elements is arranged, according to the design in Claim 13, in the housing along the longitudinal axis of the main body of the cutting device between the proximal end of the main body and the one of the two sealing elements, especially adjacent to the plane extending through the base of the one of the two sealing elements. A mutual interaction of the two sealing elements, whereby the seal can be further improved, is achieved in this way.

Of quite particular significance are furthermore the design measures of Claims 14 to 17. According to these, the one and/or the other of the two sealing elements of the sealing device is/are provided with a circular, circumferential web and/or circular, circumferential recess on its/their circumferential section facing the distal or proximal end of the main body. This/these web(s) or recess(es) can serve additionally as a sealing lip. At the same time, this/these web(s) or recess(es) enable(s) a mutual centring or centring relative to the housing element of the housing. In a particularly advantageous manner, this/these web(s) or recess(es) provide(s) additional security for both sealing elements with respect to each other and with respect to the housing element of the housing. Slipping or other displacement of the two sealing elements from the installed position to an undesirable deformation position, in which the sealing effect can no longer be maintained, is thus precluded.

Moreover, the circular, circumferential web and/or the circular, circumferential recess of the one and/or of the other of the two sealing elements of the sealing device according to Claim 18 can be preferably designed with a quadrangular cross-section, especially square or rectangular, or are U-shaped or V-shaped.

According to Claim 19, it is provided according to the invention, as an alternative or an addition, that the circular, circumferential web and/or the circular, circumferential recess of the one and/or of the other of the two sealing elements of the sealing device is designed with a shape and dimensions suitable for mutual engagement with each other or for a correspondingly shaped and dimensioned recess and/or web on the housing element of the housing. In this way, the advantages of centring, the additional safety and the increased sealing effect can be still further improved.

The advantage whereby a mutual interaction of both sealing elements can be achieved and the seal still further improved, is further reinforced by the device according to the invention in that the two sealing elements of the sealing device according to Claim 20 can be fixed to each other by a clamping disc or similar in the housing.

To improve the sealing effect the at least one, especially the two, sealing element(s) of the sealing device according to Claim 21 is/are made of flexible plastic, especially silicon.

Of particularly great significance for the device according to the invention are furthermore the features of Claim 22, according to which the sealing device for closing and opening the proximal opening at the proximal end of the main body for excising and removal of cylinders of tissue from tissue is arranged on the hollow cylindrical main body of the cutting device, which is surrounded by at least one further cutting device, with the cutting devices arranged in each other and designed to be rotatable relative to each other and also individually, with the actuating device having at least one drive device for rotating the respective main body of the at least two cutting devices about its longitudinal axis.

The design measures of Claims 23 to 25 represent advantageous embodiments of the device in accordance with the invention.

Furthermore, the features of Claims 26 to 29 serve for a particularly efficient and individually adaptable use and operation of the device according to the invention during an operation. This also means that surgery can be performed substantially more gently for a patient. The versatility of the device is thus additionally improved.

Of particular interest are the features of Claim 30. According to this, the cutting element of the cutting device, which surrounds the distal opening, is designed as essentially chamfered, tapered, conical or sloping, facing away from the distal opening and running outwards. The cutter has a very sharp wavy cut which performs a precise cut into the tissue when the main body is rotated, without the remaining tissue being excessively traumatised. The tissue can thus be removed without the surrounding tissue being deformed by the operation.

In this connection, the cutting element of the cutting device according to Claim 31 advantageously has a surrounding and/or continuous or discontinuous smooth, wavy or saw-tooth edge.

According to Claim 32, the cutting element of the cutting device is preferably fitted with a wavy edge at the foremost edge of the distal opening. This arrangement of the invention results in several circular arc sections distributed around the circumference, with wave valleys axially retreating and wave peaks axially advancing. For example, if there were four wave peaks and four wave valleys retreating, slightly axially in the circumferential area and lying in-between the four wave peaks, circular arc sections in the approximate range of 40 to 45° would result. The wavy edge can in this case be circumferential or continuous, i.e., be present over the complete circumference of the distal opening. Advantageously, the sharp zone in the transition area between the wave valley and wave peak can be designed to be particularly sharp, so that axial fixing of the tissue can be first achieved and then the morcelling of tissue cylinders or tissue sleeves carried out by the rotary movement of the main body. The wavy edge may also, however, be discontinuous in the form of an imbricated arrangement around the casing circumference at the distal opening.

The same applies for the alternative embodiment of the invention according to Claim 33. According to this, the cutting element of the cutting device with the saw-tooth edge has teeth distributed over the circumference with axially retreating tooth roots and axially advancing tooth crests. A circumferential and/or continuous or discontinuous saw-tooth edge is conceivable depending on the required application.

A substantial simplification of manufacture and assembly of the device according to the invention and an associated cost saving are provided by the measures of Claim 34, according to which the actuating device and the sealing device are integrally formed.

In this respect, the housing of the sealing device, especially the closure element of the housing, is provided, according to the technical features of Claim 35, with a profile on its circumference for the manual actuation and rotation of the main body.

In an alternative and/or additional embodiment for the manual actuation of the main body when punching out or removing tissue it is also possible to impart the rotational movement to the main body by means of a motor, which is driven electrically, pneumatically or hydraulically. For this purpose, the housing of the sealing device according to Claim 36 can be provided on its circumference with a profile, especially toothed wheels, an annular gear unit, a toothed ring or similar, in order to interact with a corresponding mating profile of a motor or a component of said motor for the automatic actuation and rotation of the main body.

According to the measures of Claim 37, the housing of the sealing device is provided on its circumference with holes or recesses and/or projections arranged radially that interact with correspondingly designed projections and/or holes or recesses on the motor or on a component of said motor.

Advantageously, the main body according to Claim 38 is provided with at least one centring and/or guide element that is arranged at a distance from the sealing device and has an external diameter that is greater than the external diameter of the main body. The external diameter of the at least one centring and/or guide element is matched to the internal diameter of a hollow cylindrical trepan sleeve of a trepan and is used for centring and guiding the main body. By means of said centring and/or guide element, a coaxial alignment of the main body of the cutting device relative to the trepan sleeve is achieved.

In a particularly advantageous manner, the housing element and/or the closure element of the housing and/or the centring and/or guide element and/or the clamping disc according to Claim 39 can be made of plastic, especially polyoximethylene, polyester, ABS, acrylic, polycarbonate, tetrafluorethylene or Impax, duroplastic elastomers with or without glass fibre reinforcement. The device according to the invention, which in this case is intended for single use, can in this way be manufactured at significant cost saving.

As an alternative to this, it is also within the framework of the invention for the main body of the cutting device and/or the housing element and/or the closure element of the housing and/or the centring and/or guide element and/or the clamping disc according to Claim 40 to be made of metal, especially (stainless) steel, high-grade steel, aluminium, brass, zinc, leaded red brass or an alloy of same. In this case, the device can be returned to multiple use after appropriate thermal and/or chemical sterilisation.

This object is finally achieved also with regard to use by the features of Claim 41.

According to this, a device can be used for excising and removing cylinders of tissue from tissue located within a body cavity or articular cavity and/or in a wall area of same, for example of an organic body at least partially held by and/or surrounded by an organic cavity of a human body, preferably for the removal of embryos, myomas, tumours, ulcers and carcinomas. The device according to the invention is particularly suitable for laparoscopic, thoracoscopic or arthroscopic and similar minimal-invasive surgical procedures. Without being shown in detail, it is also possible with the aid of the device according to the invention to excise and remove inorganic bodies such as gallstones and bladder stones or similar, which are located within a body cavity or articular cavity and/or in a wall area of same, in the form of such body cylinders.

Further features, advantages and details of the invention are given by the following description of some preferred embodiments of the invention and by the aid of drawings. The drawings are as follows:

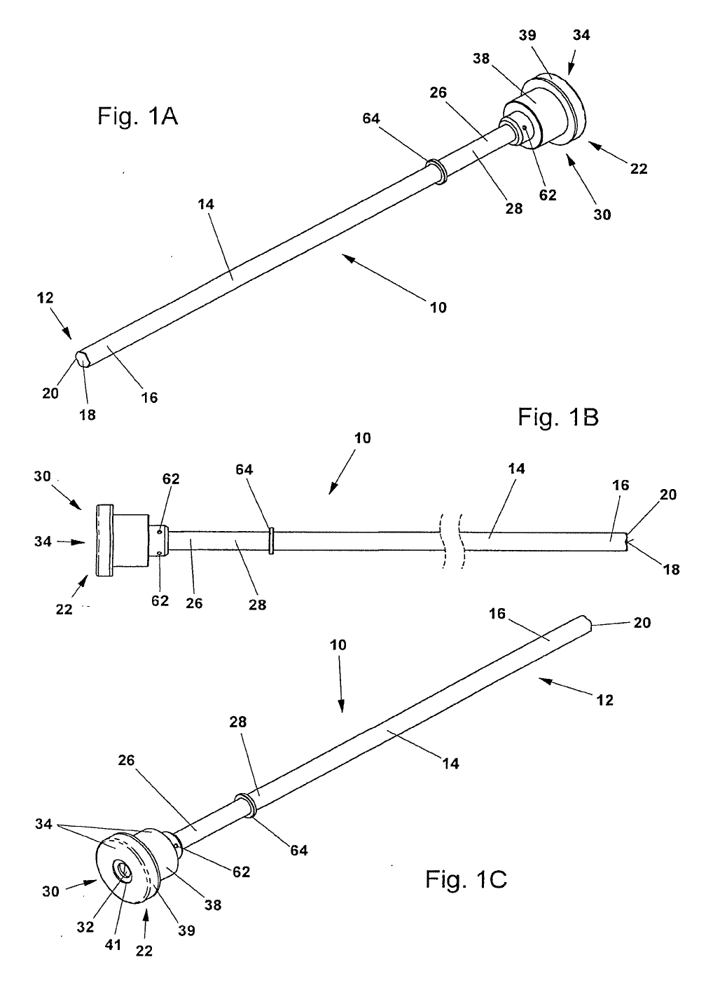

FIGS. 1A, 1B and 1C A perspective front view, a deconstructed side view and a perspective rear view of an embodiment of a device according to the invention for excising and removing cylinders of tissue from tissue.

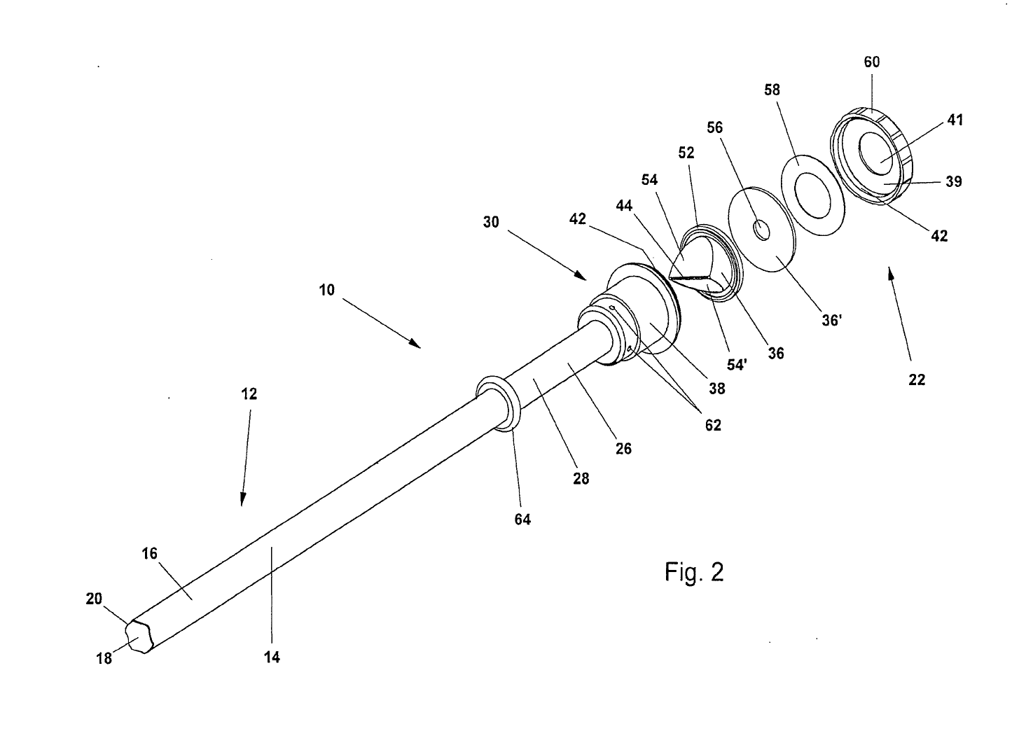

FIG. 2 An exploded perspective view of the embodiment of the device in accordance with the invention corresponding to FIG. 1A, shown enlarged.

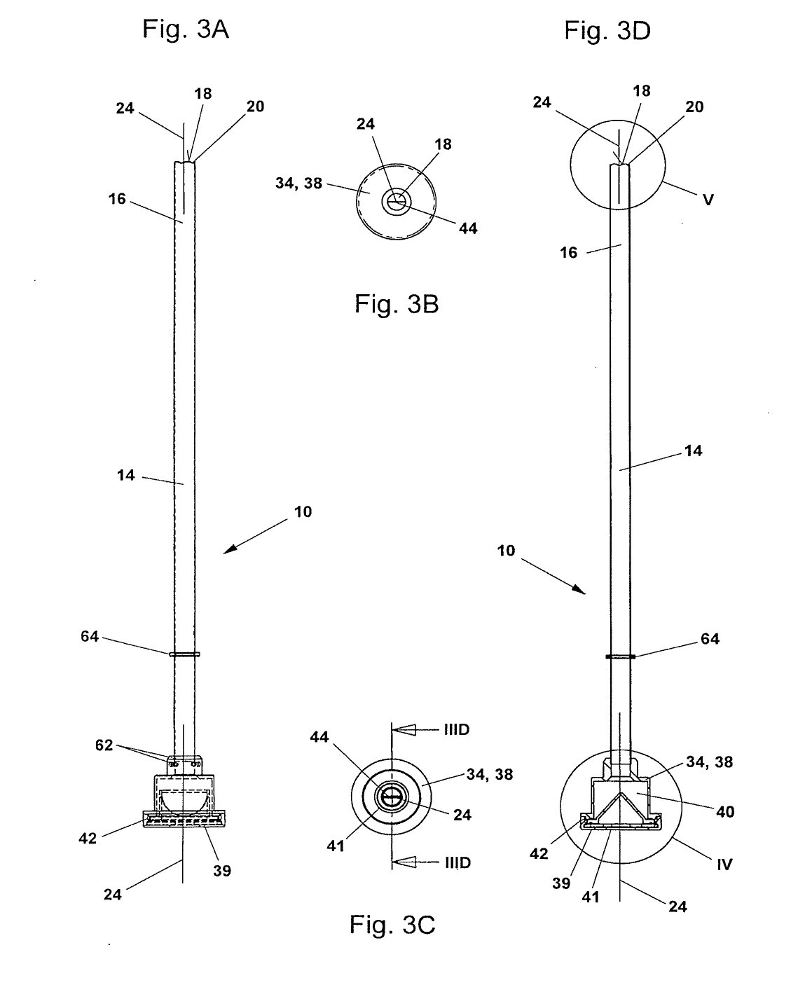

FIG. 3A to 3D A partially deconstructed, schematic side view, a front view and a rear view of the embodiment of the device in accordance with the invention corresponding to FIG. 1A to 2, and also a section view through the device in accordance with the invention corresponding to Line IIID to IIID in FIG. 3C.

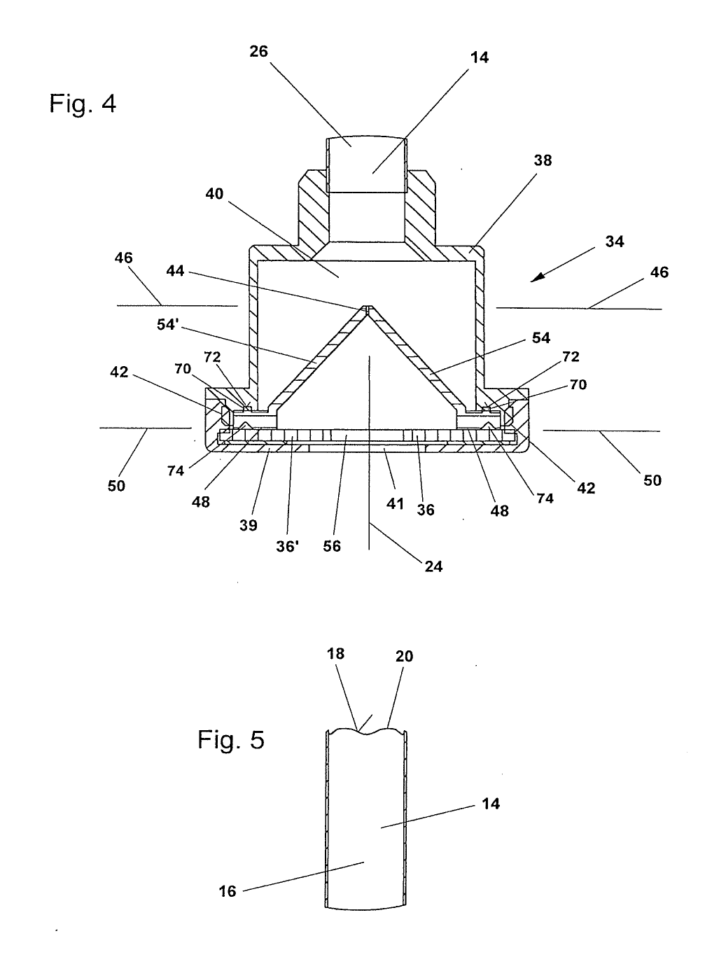

FIG. 4 A cross section view through an embodiment of a sealing device in accordance with the invention at the proximal end of the device corresponding to Section IV in FIG. 3D, shown enlarged.

FIG. 5 A cross section view through an embodiment of a cutting element in accordance with the invention at the distal end of the device corresponding to Section V in FIG. 3D, shown enlarged.

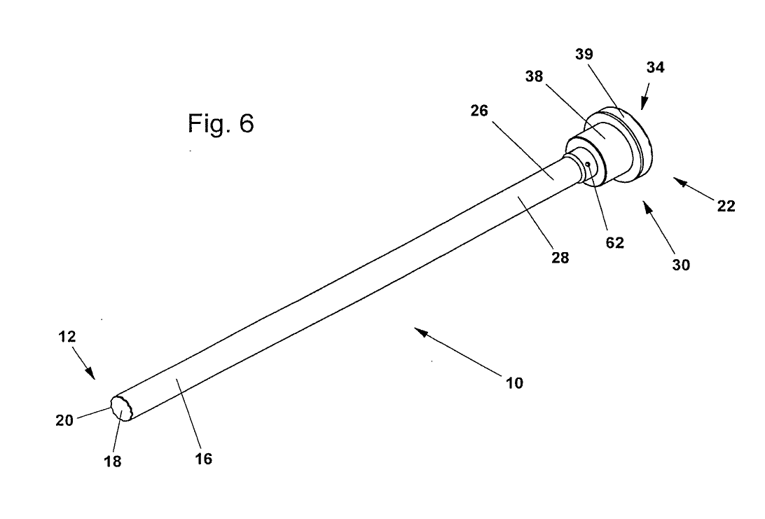

FIG. 6 A perspective front view of a further embodiment of a device in accordance with the invention for excising and removing cylinders of tissue from tissue corresponding to FIG. 1A.

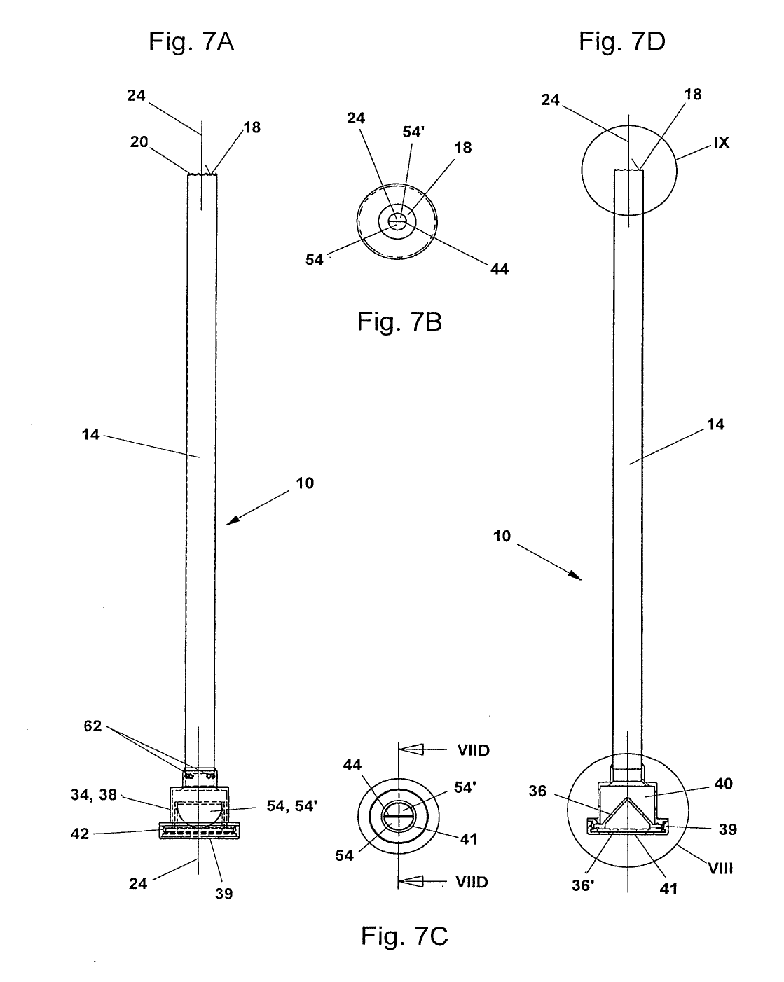

FIG. 7A to 7D A partially deconstructed, schematic side view, a front view and a rear view of the embodiment of the device in accordance with the invention corresponding to FIG. 6, and a cross-section view through the device in accordance with the invention corresponding to Line VIID to VIID in FIG. 7C.

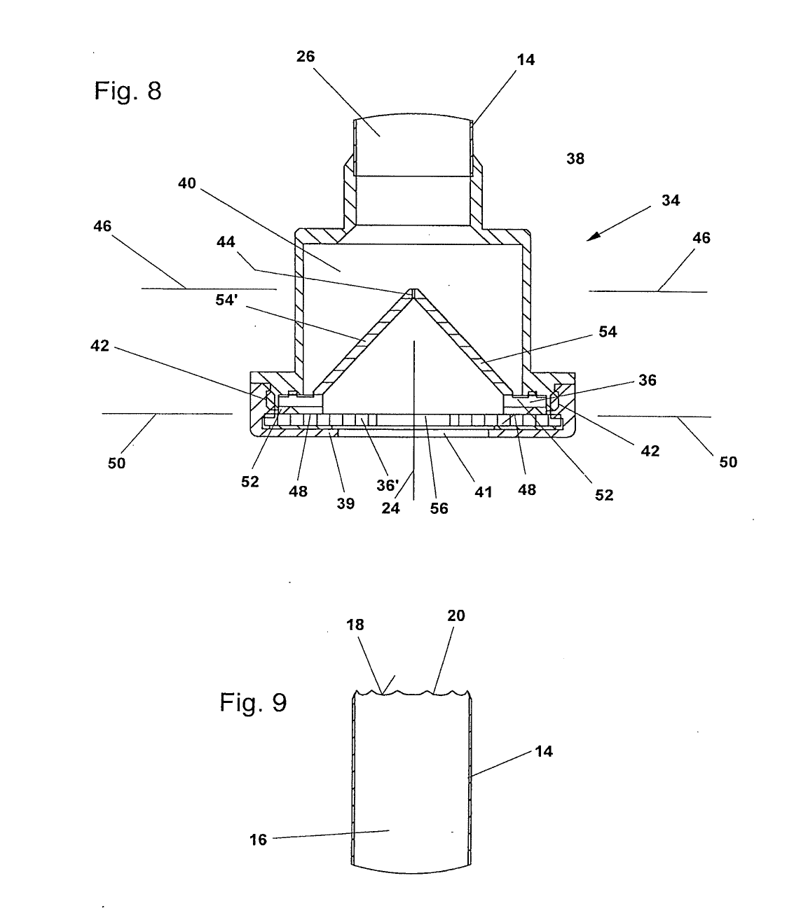

FIG. 8 A cross-section view through an embodiment of a sealing device in accordance with the invention at the proximal end of the device corresponding to Section VIII in FIG. 7D, shown enlarged.

FIG. 9 A cross-section view of an embodiment of a cutting element in accordance with the invention at the distal end of the device corresponding to Section IX in FIG. 7D, shown enlarged.

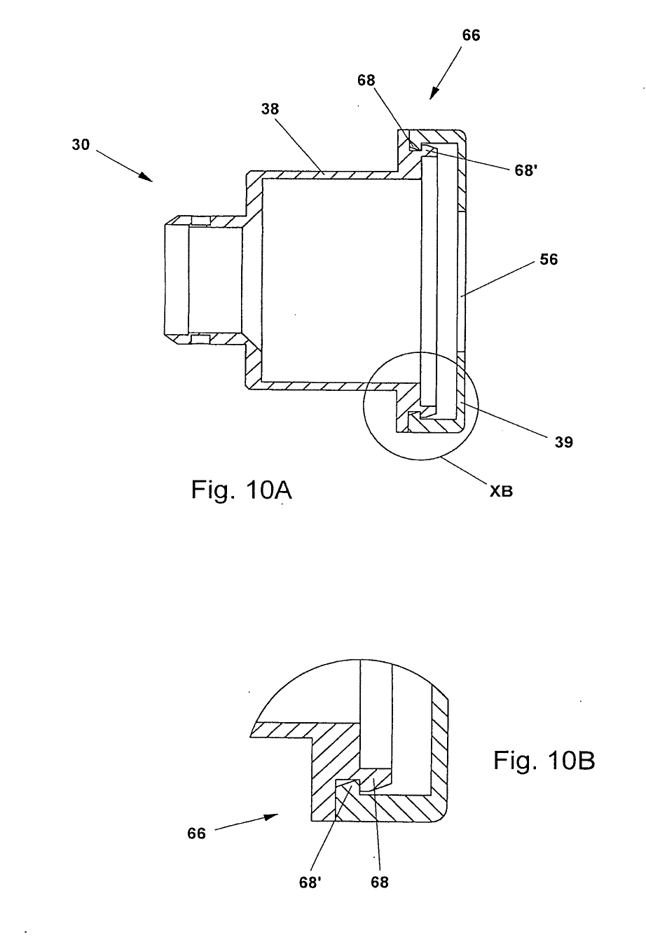

FIGS. 10 and 10B A cross-section view through a further embodiment of a sealing device in accordance with the invention at the proximal end of the device corresponding to FIGS. 4 and 8, and a cross-section view corresponding to Section XB in FIG. 10A, shown enlarged.

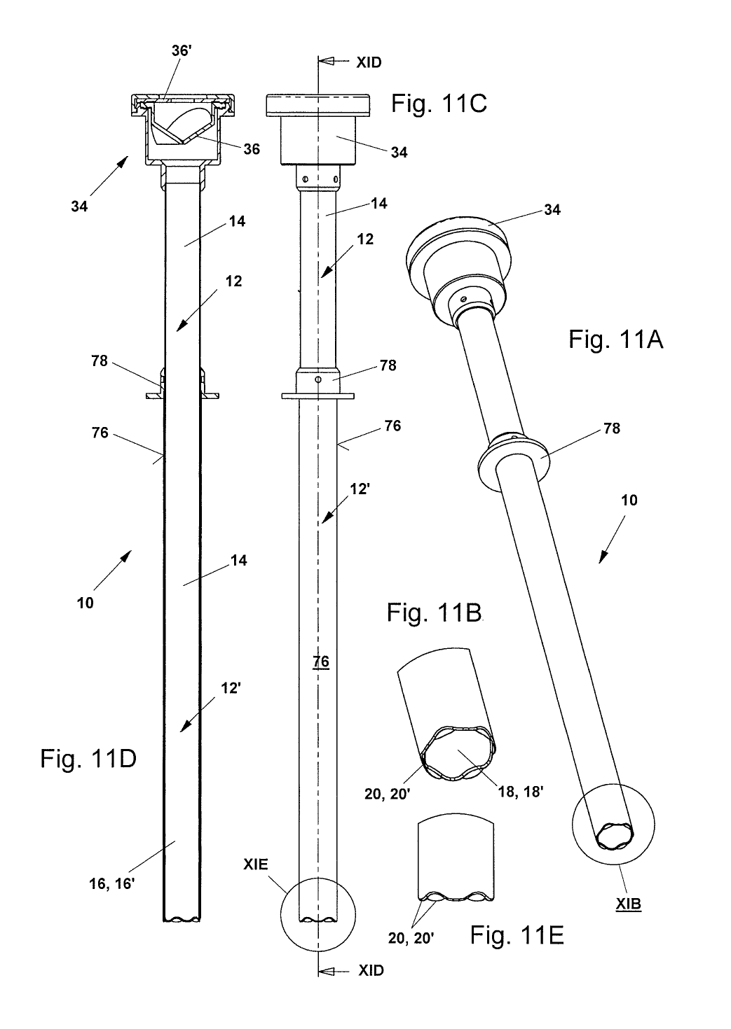

FIG. 11A to 11D A perspective view, an enlarged end view, partially discontinued perspective front view corresponding to Section XIB in FIG. 11A, a side view, a cross-section view corresponding to Line XID to XID in FIG. 11C and an enlarged end, partially discontinued, side view corresponding to Section XIE in FIG. 11C of a different embodiment of a device according to the invention.

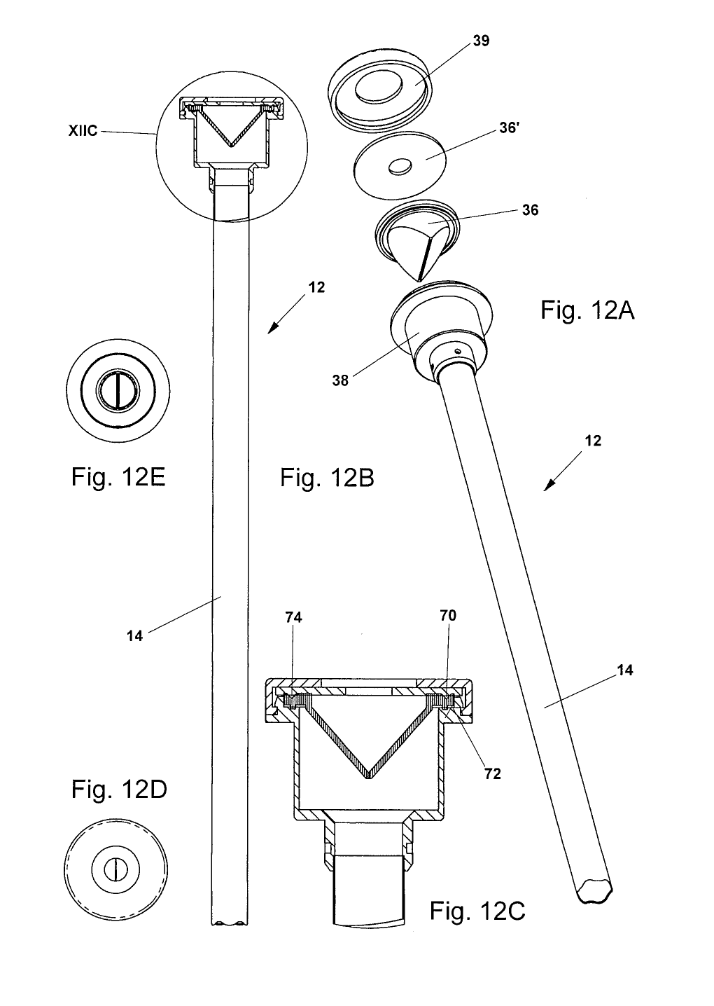

FIG. 12A to 12E An exploded, perspective front view, a partially deconstructed side view, an enlarged end, partially discontinued cross-section view corresponding to Section XIIC in FIG. 12B, a front view and a rear view of an inner cutting device of the other embodiment of the device in accordance with the invention corresponding to FIG. 11A to 11E.

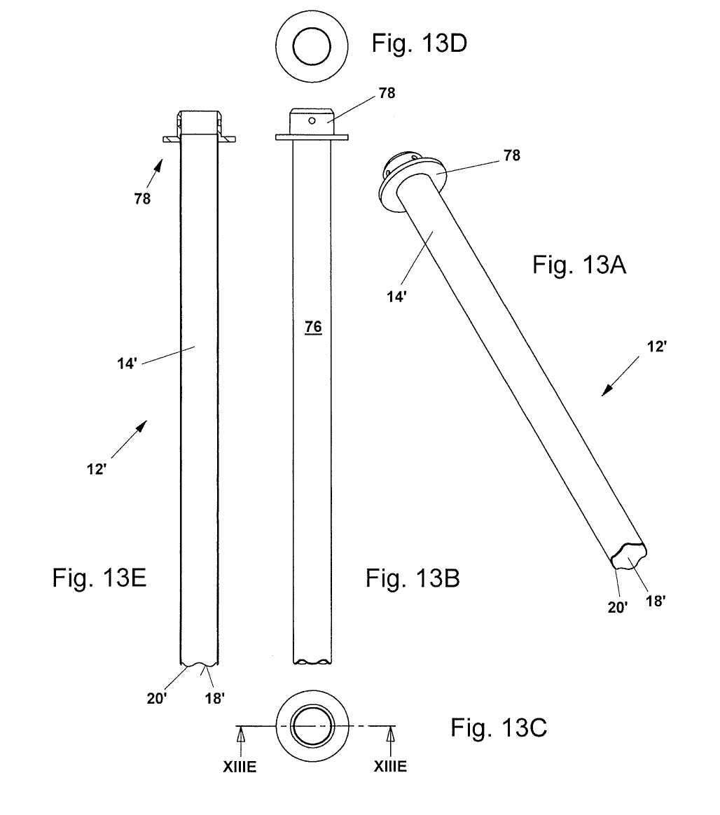

FIG. 13A to 13E A perspective front view, a side view, a front view, a rear view and a cross-section view corresponding to Line XIIIE to XIIIE in FIG. 13C through an outer cutting device of the other embodiment of the device in accordance with the invention corresponding to FIG. 11A to 11E.

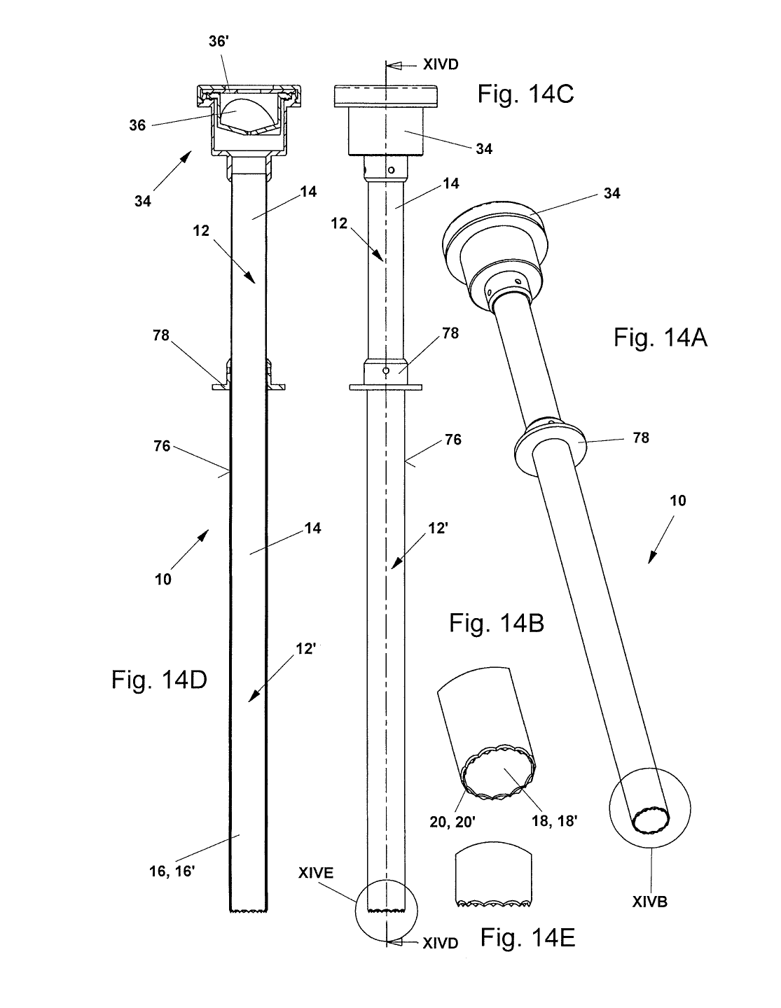

FIG. 14A to 14D A perspective view, an end enlarged, partially discontinued perspective front view corresponding to Section XIVB in FIG. 14A, a side view, a cross-section view corresponding to Line XIVD to XIVD in FIG. 14C and an end enlarged, partially discontinued side view corresponding to Section XIVE in FIG. 14C of a further different embodiment of a device in accordance with the invention.

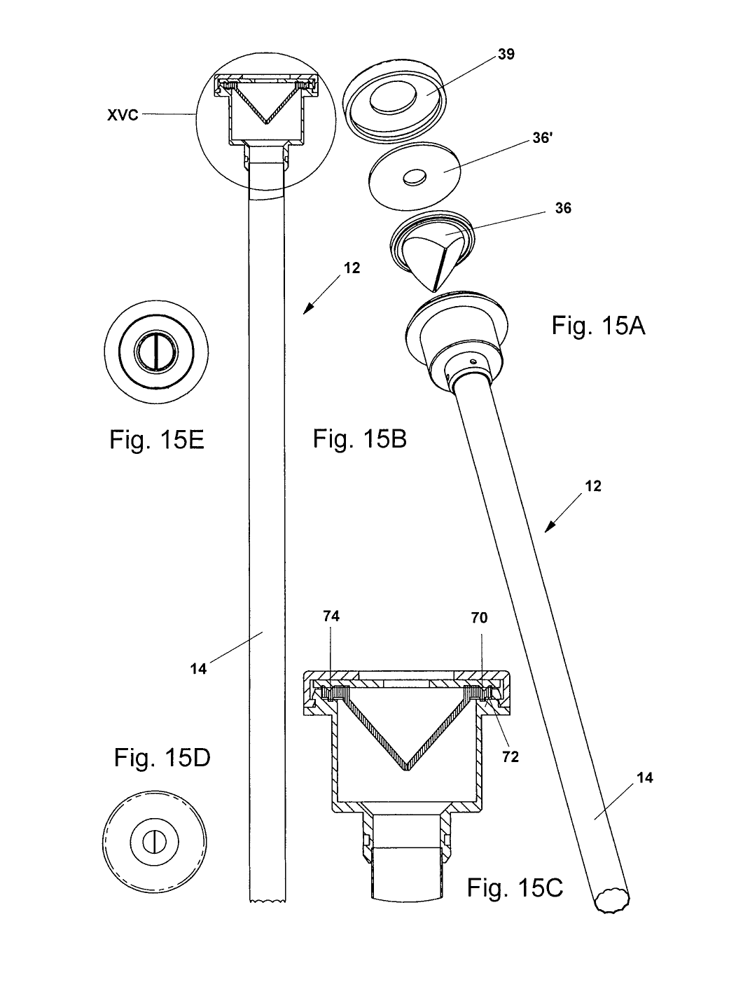

FIG. 15A to 15E An exploded, perspective front view, a partially deconstructed side view, an end enlarged, partially discontinued cross-section view corresponding to Section XVC in FIG. 15B, a front view and a rear view of an inner cutting device of the other embodiment of the device in accordance with the invention corresponding to FIG. 15A to 15E.

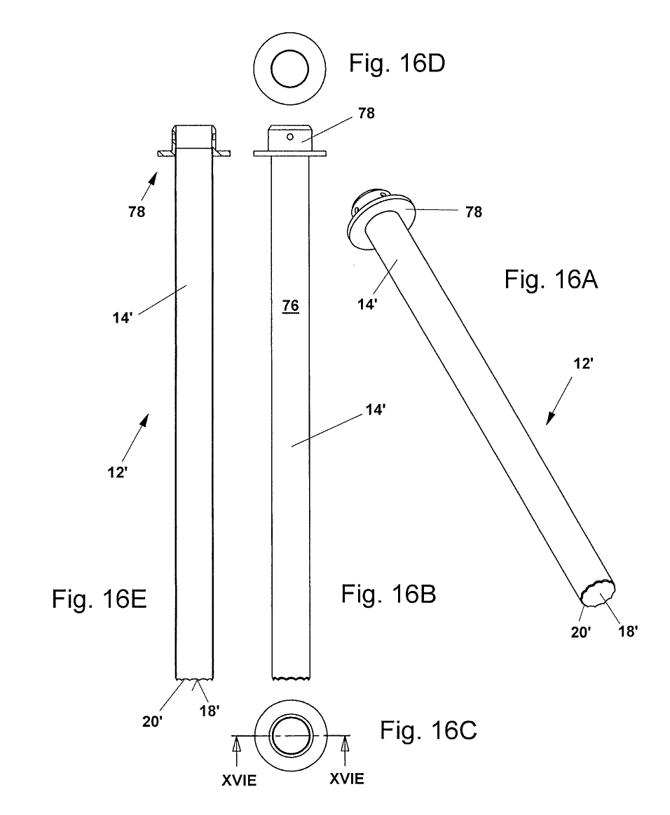

FIG. 16A to 16E A perspective front view, a side view, a front view, a rear view and a cross-section view corresponding to Line XVIE to XVIE in FIG. 16C through an outer cutting device of the other embodiment of the device in accordance with the invention corresponding to FIG. 16A to 16E.

The device 10 in accordance with the invention is provided for the excising and removal of cylinders of tissue from tissue located within a body cavity or articular cavity and/or in a wall area of same. In the following description of various examples of embodiments of the device 10 in accordance with the invention, components which are the same as each other are given identical reference characters in each case.

The device 10 according to the invention is suitable in its widest sense for operations, especially the removal of organic tissue, preferably of myomas, tumours, ulcers, carcinomas, etc., or an inorganic body, such as gallstones or bladder stones or similar agglomerations, which are at least partially held and/or surrounded by a body cavity or articular cavity or an organic cavity of a human or animal body and/or in a wall area of same.

FIGS. 1 to 5 show a first embodiment of such a device 10 according to the invention.

The device 10 includes a cutting device 12 with a hollow cylindrical main body 14. A distal opening 18 is located at the distal end 16 of the main body 14. The distal opening 18 is surrounded by the cutting element 20.

Furthermore, the device 10 is provided with an actuating device 22 which is designed for rotation of the main body 14 about its longitudinal axis 24. The actuating device 22 is arranged at a proximal end 26 of the main body 14. Alternatively or as shown in this present example of an embodiment, cumulatively, the actuating device 22 can be arranged at an area 28 of the main body 14 which merely faces towards and/or is adjacent to the proximal end 26.

Moreover, the device 10 has a sealing device 30 for closing and opening a proximal opening 32, which is located at the proximal end 26 of the main body 14. The sealing device 30 includes a housing 34 and at least one sealing element 36, 36′ which can be accommodated in the housing 34. As shown in FIGS. 1A to 5, a total of two sealing elements 36, 36′ are provided on the illustrated embodiment of the device 10 in accordance with the invention. Without being shown in detail, it is easily conceivable to merely provide a single sealing element, for example as an integral component of both illustrated sealing elements 36, 36′, or also more than two such sealing elements 36, 36′.

The housing 34 is formed from a housing element 38 and a closure element 39.

The housing element 38 is essentially pot-shaped, accommodates the at least one sealing element 36, 36′ and is unreleasable attached to the main body 14. The at least one sealing element 36, 36′ extends at least in part into a chamber 40 which is enclosed by, and therefore formed by, the housing element 38 of the housing 34.

The closure element 39 is cover- or cap-shaped. The closure element 39 is provided with a central, approximately circular bore 41 or similar opening. A further instrument (not illustrated), especially a gripping or other cutting instrument, preferably a claw gripper or myoma drill, can be passed through the bore 41, i.e., via the bore 41 through the proximal opening 32 of the main body 14, through the sealing device 30 at the proximal end 26 of the main body 14, through the main body 14 itself on to the distal end 16 of the main body 14, through the distal opening 18 finally to the (cylinder of) tissue to be removed in the operating area. The closure element 39 can in turn be attached to the housing element 38.

For this purpose, the housing element 38 and the closure element 39 of the housing 34 can be connected to each other by a threaded connection 42, and the connection can be either releasable or unreleasable. Whether it is designed with a releasable or unreleasable connection between the housing element 38 and the closure element 39 depends on the use and purpose of the device 10, i.e., whether it is intended for single (one-off) use or multiple use, the construction of the device 10 and the choice of materials from which the individual components of the device 10 in accordance with the invention are made, the wishes of the users, etc.

In the case of a single use of the device 10, the housing element 38 and the closure element 39 are unreleasable connected to each other by a threaded part with an adhesive agent, preferably a silicone adhesive, arranged in between. In this way, a thermal or chemical sterilisation of the device 10 and any repeated or further use of the device 10 are ruled out.

With the embodiment shown in FIGS. 1A to 5, the sealing device 30 includes, as already mentioned, two sealing elements 36, 36′, which are arranged adjacent to each other in the housing on the longitudinal axis 24 of the main body 14 of the cutting device 12 and interact with each other.

One 36 of the two sealing elements 36, 36′ of the sealing device 30 is essentially designed in the shape of a cone, a truncated cone, a pot, a hat or similar. The sealing element 36 has a slotted opening 44 that is arranged in a plane 46 (vertical to the sheet plane), which is offset in the direction of the distal end 16 of the main body 14 with respect to a plane 50 (vertical to the sheet plane) extending to the base 48 of the sealing element 36. The sealing element 36 therefore extends with the part or section essentially designed in the shape of a cone, a truncated cone, a pot, a hat or similar, in the direction of the distal end 16 of the main body 14 and projects into the chamber 40 of the housing 34. Furthermore, the sealing element 36 has a circumferential section 52 extending radially outwards in the plane 50 that extends through the base 48 of the sealing element 36 and serves for fixing in the housing 34.

The sealing element 36 has at least two sealing lips 54, 54′ that are in mutual effective engagement, to close and open the slotted opening 44. Due to the shape and arrangement of the two sealing lips 54, 54′ of the sealing element 36 in the housing 34 in an opposing direction to the action of a pressure artificially created inside a body cavity or articular cavity, for example due to CO₂ insufflation, the sealing lips 54, 54′ seat tightly on the outside against a further instrument (not illustrated), for example a gripping or other cutting instrument, especially a claw gripper or a myoma drill, and form a complete seal. In this way, a virtual wedging of the sealing lips 54, 54′ takes place. The sealing lips 54, 54′ are pressed against the further instrument and remain in this position until there is a controlled release of the pressure. In this way, it is guaranteed that the pressure which is necessary for operating, i.e., excising or punching out a cylinder of tissue from tissue and then removing the excised or punched out cylinder of tissue from the operating area, is permanently maintained. An escape of gas such as CO₂ or similar from the body cavity or articular cavity of the operating area is effectively prevented.

Another 36′ of the two sealing elements 36, 36′ of the sealing device 30 is designed in the form of a disc and is provided with a central, approximately circular opening 56. Said further instrument (not illustrated), especially a gripping or other cutting instrument, preferably a claw gripper or myoma borer, can be inserted through the opening 56 of the sealing element 36′. The central, approximately circular opening 56 is provided with an internal diameter which is smaller or equal to an external diameter of the further instrument.

As can be seen from FIGS. 2, 3D and 4, the other sealing element 36′ is arranged in the housing 34 on the longitudinal axis 24 of the main body 14 between the proximal end 26 of the main body 14 and the one sealing element 36. In particular, the other sealing element 36′ is arranged (immediately) adjacent to the plane 50 extending to the base 48 of the one sealing element 36.

The two sealing elements 36, 36′ of the sealing device 30 can be fixed to each other by a clamping disc 58 or similar in the housing 34.

According to FIGS. 1A to 3A, 3D and especially 5, with the embodiment of the device 10 the cutting element 20 of the cutting device 30 is designed as essentially chamfered, tapered, conical or obliquely facing away from the distal opening 18 and running outwards. In this case, the cutting element 20 can have a surrounding or discontinuous smooth, wavy or saw-toothed edge.

With the embodiment of the device 10 shown in FIGS. 1A to 3A, 3D and 5, the cutting element 20 is provided with a wavy edge with the wavy edge having circular arc sections distributed around the circumference, with wave valleys axially retreating and wave peaks axially advancing.

As can also be seen from FIGS. 1A to 3A, the actuating device 22 and sealing device 30 are integrally formed in an advantageous manner.

Moreover, the housing 34 of the sealing device 30 and in particular the closure element 39 of the housing 34 are provided with a profile 60 on its circumference for the manual actuation and rotation of the main body 14.

In an alternative and/or cumulative embodiment, the housing 34 of the sealing device 30 can be provided with a profile on its circumference for interaction with a corresponding mating profile of a motor or a component of said motor (neither illustrated) for the automatic activation and rotation of the main body 14. Toothed wheels, an annular gear unit or a toothed ring, etc., are suitable for this purpose. With the embodiment presented in FIGS. 1A to 5, the housing 34 is, for example, provided with holes 62 or recesses or projections (not illustrated) arranged radially on its circumference, that interact with correspondingly designed projections and/or holes or recesses on the motor or on a component of said motor.

Finally, the embodiment of the device 10, which is shown in FIGS. 1A to 5, is fitted with at least one centring and/or guide element 64. The centring and/or guide element 64 is arranged at or on the main body 14 at a distance from the sealing device 30. The external diameter of the centring and/or guide element 64 is greater than the external diameter of the main body 14 and corresponds approximately to the internal diameter of a trepan sleeve (not illustrated) in which the main body 14 is mounted, so that it can be rotated and slid backwards and forwards.

The other embodiment of the device 10 according to the invention shown in FIGS. 6 to 9 is largely the same as that shown in FIGS. 1A to 5.

The embodiment of the device 10 according to FIGS. 6 to 9 differs from the previously described embodiment only in that the diameter of the main body 14 is larger. The main body 14 of the device 10 can thus have various diameters, for example of 10 mm to 30 mm and a length preferably of 30 cm. If the external diameter of the main body 14 corresponds approximately to the internal diameter of the trepan sleeve, the device 10 then does not require a centring and/or guide element 64.

A basic difference, however, is in the design of the cutting element 20 of the cutting device 30, which is provided with a saw-tooth edge. Therefore, the cutting element 20 has teeth distributed around the circumference with axially retreating tooth roots and axially advancing tooth crests.

Finally, another different embodiment of a sealing device 30 according to the invention is shown in FIGS. 10A and 10B. According to this, a latching or snap-in connection 66 can be used instead of a threaded connection for the mutual attachment of the housing element 38 and closure element 39. In this case, the housing element 38 and the closure element 39 are connected to each other by at least one latching projection 68 or similar latching lug on the housing element 38 or on the closure element 39 and at least one further latching projection 68′ on the closure element 39 or on the housing element 38. Both latching projections 68, 68′ or similar latching lugs interact with each other and can be brought into mutual engagement.

Preferably the at least one latching projection 68 or similar latching lug and/or the at least one further latching projection 68′ or similar latching lug can be designed in this connection in such a way that the housing element 38 and the closure element 39 of the housing 34 are unreleasably connected to each other after being brought into engagement with each other or cannot be connected to each other following subsequent disengagement with each other. For example, one or both of the latching projections 68, 68′ or similar latching lug(s) can be provided with a design breakpoint, in order to permanently damage the device 10 as it is disengaged and thus make it unfit for further use.

The at least one, especially the two sealing element(s) 36, 36′ of the sealing device 30 is/are made of flexible plastic, especially silicone.

Referring back to the embodiment of the device according to the invention in FIG. 4, the one sealing element 36 of the two sealing elements 36, 36′ of the sealing device 30 has a circular, circumferential web 70. The web or similar projection is arranged on the circumferential section 52 of the sealing element 36. With the embodiment of the device 10 shown in FIG. 4, the web 70 is integrally formed on the circumferential section 52, which is facing towards the distal end 16 of the main body 14.

The circular, circumferential web 70 of the one 36 of the two sealing elements 36, 36′ is, as clearly shown in FIG. 4, designed to have a cross section which is approximately quadrangular, especially square or rectangular. As an alternative, a different cross section can, however, be used, for example a U-shaped or V-shaped cross section.

According to FIG. 4, the circular, circumferential web 70 of the one sealing element 36 interacts with a recess 72 which is arranged on the housing element 38 of the housing 34. The web 70 and recess 72 are matched to each other with regard to shape and dimensions, in order to mutually engage as a form fit or force fit. The web 70 and the recess serve in this way as an additional sealing lip or sealing barrier. At the same time, a mutual centring or a centring with respect to the housing element 38 of the housing 34 is achieved. An additional security of the sealing element 36 with respect to the housing element 38 is also obtained. Slipping or other displacement of the sealing element 36 from the installed position to an undesirable deformed position, in which the sealing effect can no longer be maintained, is thus precluded.

Without being shown in detail, a kinematic reversal of the web 70 and recess 72 can equally be provided. With such a design, a circular, circumferential recess is provided on the circumferential section 52, which is facing towards the distal end 16 of the main body 14. Opposite this, a correspondingly formed circular, circumferential web would be formed on the housing element 38 of the housing 34.

Finally, it would also be conceivable, without also being shown in detail, for the recess 72 on the housing element or a web provided as an alternative to be completely omitted because the one sealing element has a greater deformability than the housing element. However, the previously indicated advantages could not be achieved by such an arrangement like in the exemplary embodiment according to FIG. 4.

Furthermore, as shown in FIG. 4, the one sealing element 36 of the two sealing elements 36, 36′ of the sealing device 30 is provided with a circular, circumferential recess 74 on its circumferential section 52, which faces towards the proximal end 26 of the main body 14. The recess 74 in this case is designed to be approximately V-shaped, but equally can have a cross section in the form of a quadrangle, especially a square or a rectangle, or be U-shaped. The function of the recess 74 with regard to centring, securing the sealing element 36, slipping or other displacement of the sealing element 36 from the installed position to an undesirable deformed position and the sealing effect is the same as that which has already been explained in conjunction with the web 70.

Accordingly, the recess 74 can interact with a correspondingly shaped circular, circumferential web (not illustrated in this example), which is arranged on the other sealing element 36′ of the two sealing elements 36, 36′ of the sealing device 30, on its outer edge which faces towards the distal end 16 of the main body 14.

Alternatively, a kinematic reversal of the recess 74 on the one sealing element 36 and a web or a smooth surface on the other sealing element 36′ would be equally possible.

Again, without being shown in detail, it would be equally conceivable for the other sealing element 36′ of the two sealing elements 36, 36′ of the sealing device 30 to have a circular, circumferential web and/or a circular, circumferential recess on its circumferential section which faces towards the proximal end 26 of the main body 14, in order to interact or mutually engage with a corresponding recess or corresponding web on the clamping disc 58 or directly on the cover-shaped or cap-shaped closure element 39 of the housing 34.

FIGS. 11A to 13E show another embodiment of the device 10 according to the invention. The device 10 shown in FIGS. 11A to 11E differs essentially from that of the preceding embodiment in FIGS. 1A to 5 in that two cutting devices 12, 12′ are provided. Accordingly, the cutting device 12, as it is when in FIGS. 12A to 12E, is surrounded by at least one further cutting device 12′, as is shown in FIGS. 13A to 13E. Both cutting devices 12, 12′ are arranged within each other and designed so that they can be rotated relative to each other and also individually.

The inner cutting device 12 is coaxially housed, especially with a slight clearance, within the adjacent outer cutting device 12′ of the at least two cutting devices 12, 12′. If the inner cutting device 12 is housed within the adjacent outer cutting device 12′ of the at least two cutting devices 12, 12′ with a greater clearance, at least one centring and/or guide element (not illustrated) is arranged between the main bodies 14, 14′ of the inner cutting device 12 and the adjacent outer cutting device 12′.

The sealing device 30 for closing and opening the proximal opening 32 is, for excising and removing cylinders of tissue from tissue, arranged at the proximal end 26 of the main body on the hollow cylindrical main body 14 of the cutting device 12.

In addition, the main body 14′ of the cutting device 12′ is connected at its outer circumference 76 and at the same time at its proximal end 26′ with a drive sleeve 78 or similar, so that it rotates with it.

A distal opening 18, 18′ is provided at each of the distal ends 16, 16′ of the main bodies 14, 14′. The distal opening 18′ is surrounded by a cutting element 20, 20′ which each is provided with a wavy edge. A smooth edge would be equally conceivable as an alternative.

The actuating device is designed in the form of at least one drive device (not illustrated in more detail) for rotating the respective main body 14, 14′ of the at least two cutting devices 12, 12′ about its longitudinal axis. The at least two cutting devices 12, 12′ can be driven in a contra-rotating direction and/or in the same direction relative to each other by the drive device.

A gear unit (also not illustrated), fitted within a housing, can be arranged between the at least two cutting devices 12, 12′ and the drive device. The drive device can be controlled (open-loop or closed-loop control) by means of an, especially electronic, controller, in such a way that the two cutting devices 12, 12′ can be driven by the drive device 22 at intervals in an opposing drive direction in each case. The time intervals for driving the two cutting devices 12, 12′ can be set and/or preset by an, especially electronic, controller, in order to drive the cutting devices in opposing directions.

In other respects, the embodiment in FIGS. 11A to 13E is the same as that shown in FIGS. 1A to 5.

The embodiment of the device 10 according to the invention, which is shown in FIGS. 14A to 16E, corresponds approximately to that in FIGS. 11A to 13E. The difference, however, is in the design of the cutting elements 20, 20′, which are not designed with a wavy edge, but instead have a saw-toothed edge, i.e., are approximately congruent with the embodiment according to FIGS. 6 to 9.

The housing element 38 and/or the closure element 39 of the housing 34 and/or the clamping disk 58 and/or the centring and/or guide element 64 is/are made of plastic, especially polyoximethylene, polyester, ABS, acrylic, polycarbonate, tetrafluroethylene or Impax, duroplastic elastomers, with or without glass fibre reinforcement. Instead of these, the housing element 38 and/or the closure element 39 of the housing 34 and/or the clamping disc 58 and/or the centring and/or guide element 64 can alternative and/or cumulatively be made of metal, especially (stainless) steel, high-grade steel, aluminium, brass, zinc, leaded red brass or an alloy of same. In contrast, the main body 14 of the cutting device 12 is generally preferably made of metal, especially (stainless) steel, high-grade steel, aluminium, brass, zinc, leaded red brass or an alloy of same.

The invention is not restricted to the embodiments of the device 10 shown in FIGS. 1 to 10B. It is thus possible to modify the latching or snap-in connection 66 for mutual attachment of the housing element 38 and closure element 39 so that instead of one of the two latching projections 68 or 68′ or similar latching lug(s) a latching recess (not illustrated) or similar is provided in which the other of the two latching projections 68′ or 68 engages and unreleasably, i.e., only by destruction of the latching or snap-in connection, latches.

1-41. (canceled)

42. Device for excising and removing cylinders of tissue from tissue located within a body cavity or articular cavity and/or in a wall area of same, that includes a cutting device (12) with a hollow cylindrical main body (14), a distal opening (18) at a distal end (16) of the main body (14) and a cutting element (20) surrounding the distal opening (18), an actuating device (22) at a proximal end (26) and/or at an area (28) of the main body (14) facing towards the proximal end (14) for rotating the main body (14) about the longitudinal axis (24) of said main body (14) and a closure device (30) for closing and opening a proximal opening (32) at the proximal end (26) of the main body (14), characterized in that the sealing device (30) comprising a housing (34) which has a housing element (38) being essentially pot-shaped and attached to the main body (14) and a cover- or cap-shaped closure element (39) that can be attached to the housing element (38), and two sealing elements (36, 36′) which can be housed in the housing (34), which are arranged adjacent to each other in the housing (34) along the longitudinal axis (24) of the main body (14) of the cutting device (12) and interact with each other, with the one sealing element (36) being essentially of cone, truncated cone, pot, hat or similar shape and provided with a circular section (52) extending radially outwards, with the other sealing element (36′) being disc shaped, with the one sealing element (36) having a circular, circumferential web (70) or a circular, circumferential recess on its circumferential section (52) and the housing element (38) having a suitably shaped recess (72) or web facing the web (70) or recess for engagement with each other and/or with the other sealing element (36′) having a circular, circumferential web or circular, circumferential recess (74) on its outer border area and the one sealing element (36) having a suitably shaped recess or web facing the web (70) or recess for mutual engagement with each other and/or with the other sealing element (36′) having a circular, circumferential web or circular, circumferential recess on its outer border area and the cover- or cap-shaped closure element (39) of the housing (34) having a suitably shaped recess or web facing the web or recess for mutual engagement with each other.

43. Device according to claim 42, characterized in that the housing element (38) and the closure element (39) of the housing (34) are releasably or unreleasably connected to each other by a threaded connection (42) or a latching or snap-in connection (66).

44. Device according to claim 42, characterized in that the housing element (38) and the closure element (39) of the housing (34) are unreleasably connected to each other by a threaded part with an adhesive agent, preferably a silicone adhesive, arranged in between.

45. Device according to claim 42, characterized in that the housing element (38) and the closure element (39) of the housing (34) are connected to each other through at least one latching projection (68) or similar latching lug on the housing element (38) or on the closure element (39) and at least one other latching projection (68′) or similar latching lug or at least one latching recess or similar on the closure element (39) and/or on the housing element (38) that can interact with each other and can be brought into mutual engagement.

46. Device according to claim 42, characterized in that the at least one latching projection (68) or similar latching lug on the housing element (38) and/or on the closure element (39) and the at least one other latching projection (68′) or similar latching lug and/or the at least one latching recess or similar on the closure element (39) and/or on the housing element (38) is/are designed in such a way that the housing element (38) and the closure element (39) of the housing (34) are unreleasably connected to each other after the at least one latching projection (68) or similar latching lug and the at least one other latching projection (68′) or similar latching lug and/or the at least one latching recess have been brought into engagement with each other, or in that they cannot be connected to each other after the at least one latching projection (68) or similar latching lug and the at least one other latching projection (68′) or similar latching lug and/or the at least one latching recess have been disengaged from each other.

47. Device according to claim 42, characterized in that the one (36) of the two sealing elements (36, 36′) of the sealing device (30) has a slotted opening (44) that is arranged in a plane (46) which is offset in the direction of the distal end (16) of the main body (14) of the cutting device (12) with respect to a plane (50) extending through the base (48) of the sealing element (36).

48. Device according to claim 42, characterized in that the one (36) of the two sealing elements (36, 36′) has at least two sealing lips (54, 54′) that are in mutual effective engagement to close and open the slotted opening (44).

49. Device according to claim 42, characterized in that the other (36′) of the two sealing elements (36, 36′) of the sealing device (30) has a central, approximately circular, opening (56) through which a further instrument, especially a gripping instrument or other cutting instrument, preferably a claw gripper or a myoma drill, can be inserted.

50. Device according to claim 42, characterized in that the central, approximately circular, opening (56) of the other (36′) of the two sealing elements (36, 36′) has an internal diameter that is smaller than or equal to an external diameter of another instrument, especially a gripping instrument or other cutting instrument, preferably a claw gripper or a myoma drill, which can be inserted through the opening (56).

51. Device according to claim 42, characterized in that the other (36) of the two sealing elements (36, 36′) is arranged in the housing (34) on the longitudinal axis (24) of the main body (14) of the cutting device (12) between the proximal end (26) of the main body (14) and the one (36) of the two sealing elements (36, 36′), especially adjacent to the plane (50) extending through the base (48) of the one (36) of the two sealing elements (36, 36′).

52. Device according to claim 42, characterized in that the circular, circumferential web (70) and/or the circular, circumferential recess (74) of the one (36) and/or the other (36′) of the two sealing elements (36, 36′) of the sealing device (30) has/have a cross section which is a quadrangle, especially square or rectangular, or is/are U-shaped or V-shaped.

53. Device according to claim 42, characterized in that the circular, circumferential web (70) and/or the circular, circumferential recess (74) of the one (36) and/or of the other (36′) of the two sealing elements (36, 36′) of the sealing device (30) is/are designed, for mutual engagement, to match each other with respect to shape and dimensions, or a correspondingly shaped and dimensioned recess (72) and/or web on the housing element (38) of the housing (34).

54. Device according to claim 42, characterized in that the two sealing elements (36, 36′) of the sealing device (30) can be fixed to each other by a clamping disc (58) or similar in the housing (34).

55. Device according to claim 42, characterized in that the at least one, especially the two, sealing element(s) (36, 36′) of the sealing device (30) is/are made of flexible plastic, especially silicon.

56. Device according to claim 42, characterized in that the sealing device (30) for closing and opening the proximal opening (32) is arranged, at the proximal end (26) of the main body (14), on the hollow cylindrical main body (14) of the cutting device (12), for excising and removing cylinders of tissue from tissue, with the proximal opening being surrounded by at least one further cutting device (12′) and said cutting devices being arranged within each other and designed to be rotatable relative to each other and also individually, and having at least one drive device for rotating the respective main body (14, 14′) of the at least two cutting devices (12, 12′) about its longitudinal axis.

57. Device according to claim 42, characterized in that the inner cutting device (12) is accommodated coaxially in the adjacent outer cutting device (12′) of the at least two cutting devices (12, 12′).

58. Device according to claim 42, characterized in that the inner cutting device (12) is accommodated with a slight clearance within the adjacent outer cutting device (12′) of the at least two cutting devices (12, 12′).

59. Device according to claim 42, characterized in that the inner cutting device (12) is accommodated with a greater clearance within the adjacent outer cutting device (12′) of the at least two cutting devices (12, 12′), with at least a centering and/or guide element being arranged between the main bodies (14, 14′) of the inner cutting device (12) and the adjacent outer cutting device (12′).

60. Device according to claim 42, characterized in that the at least two cutting devices (12, 12′) can be driven by a drive device, in an opposite direction and/or in the same direction relative to each other.

61. Device according to claim 42, characterized in that a gear unit fitted in a housing is arranged between the at least two cutting devices (12, 12′) and the drive device.

62. Device according to claim 42, characterized in that the drive device can be controlled (closed-circuit control or open-circuit control) by a, especially electronic, controller in such a way that the two cutting devices (12, 12′) can be driven by the drive device (22) at intervals in each case in the opposite drive direction.

63. Device according to claim 42, characterized in that the time intervals for driving the two cutting devices (12, 12′) in an opposite drive direction can be set and/or preset as required by a, especially electronic, controller.

64. Device according to claim 42, characterized in that the cutting element (20) of the cutting device (12) is designed as essentially chamfered, tapered, conical or sloping, facing away from the distal opening (18) and running outwards.

65. Device according to claim 42, characterized in that the cutting element (20) of the cutting device (12) has a surrounding or discontinuous smooth, wavy or saw-tooth edge.

66. Device according to claim 42, characterized in that the cutting element (20) of the cutting device (12) with the wavy edge has circular arc sections distributed around the circumference, with wave valleys axially retreating and wave peaks axially advancing.

67. Device according to claim 42, characterized in that the cutting element (20) of the cutting device (12) with the saw-tooth edge has teeth distributed around the circumference, with axially retreating tooth roots and axially advancing tooth crests.

68. Device according to claim 42, characterized in that the actuating device (22) and the sealing device (30) are integrally formed.

69. Device according to claim 42, characterized in that the housing (34) of the sealing device (30), especially the closure element (39) of the housing (34), is provided with a profile (60) on its circumference for the manual actuation and rotation of the main body (14).

70. Device according to one of claim 42, characterized in that the housing (34) of the sealing device (30) is provided with a profile on its circumference, especially toothed wheels or a toothed ring, for interaction with a corresponding mating profile of a motor or a component of said motor for the automatic actuation and rotation of the main body (14).

71. Device according to claim 42, characterized in that the housing (34) of the sealing device (30) is provided with holes (62) or recesses and/or projections arranged radially around the circumference of said housing (34) that interact with correspondingly designed projections and/or holes or recesses on the motor or on a component of said motor.

72. Device according to claim 42, characterized in that the main body (14) is provided with at least one centering and/or guide element (64) that is arranged at a distance from the sealing device (30) and has an external diameter that is greater than the external diameter of the main body (14).

73. Device according to claim 42, characterized in that the housing element (38) and/or the closure element (39) of the housing (34) and/or the clamping disc (58) and/or the centering and/or guide element (64) is/are made of plastic, especially polyoximethylene, polyester, ABS, acrylic, polycarbonate, tetrafluorethylene or Impax, duroplastic elastomers, with or without glass fiber reinforcement.

74. Device according to claim 42, characterized in that the main body (14) of the cutting device (12) and/or the housing element (38) and/or the closure element (39) of the housing (34) and/or the clamping disc (58) and/or the centering and/or guide element (64) is/are made of metal, especially (stainless) steel, high-grade steel, aluminum,
